(19) United States
(12) Patent Application Publication
(10) Pub. No.: US 2004/0097946 A1
(43) Pub. Date: May 20, 2004

(54) Device for cutting bones to size

(75) Inventors: Daniel Dietzel, Kelkheim (DE); Helmut Heckele, Knittlingen (DE)

Correspondence Address: Thomas C. Pontani, Esq., Cohen, Pontani, Lieberman & Pavane, Suite 1210, 551 Fifth Avenue, New York, NY, 10176, US

(73) Assignee: Richard Wolf GmbH

(21) Appl. No.: 10/628,013

(22) Filed: Jul. 25, 2003

(30) Foreign Application Priority Data
Jul. 25, 2002 (DE) 102 33 808.6

(51) Int. Cl. A61B 17/16

(52) U.S. Cl. 606/79

(57) ABSTRACT

The invention relates to a device for cutting bones to size, in particular with displacement osteotomy, with at least one mounting part in which there is formed a receiving channel for receiving a bone piece. In the mounting pat in the region of the receiving channel there is formed at least one slot arranged at an angle to the longitudinal axis of the receiving channel.

### BACKGROUND OF THE INVENTION

The invention relates to a device for cutting bones to size, in particular with displacement osteotomy.

There are known operative methods in order to correct malposition of the legs, such as knock-knees and/or bowlegs. For this, with known methods one drills radially several times at a certain distance through the bones at defined locations along a circumferential line. In this manner one creates a weakened location in the bone which permits the adjacent bone sections to be brought into a somewhat extended position. For fixing these aligned bone sections one requires external fixing means which have to be removed again by operation after the two bone sections have grown together again.

### BRIEF SUMMARY OF THE INVENTION

It is the object of the invention to provide a device which permits an improved operation method for correcting bone malposition. This object is achieved by a device with the features specified in claim 1. Preferred embodiment forms are to be deduced from the dependent claims.

The invention is based on a new operation method for alleviating knock-knees and/or bowlegs, so-called wedge osteotomy. With this method a bone to be corrected is sawn to a certain depth at a suitable location. In order then to align the bone in a more or less straight position one introduces at least one bone wedge created from the body's own bone material into the sawn gap which has been previously incorporated into the bone. These bone wedges may have different thickness and wedge angles, according to the extent of the correction to be effected. This method has the advantage that the sawn bone grows quickly together again. For manufacturing the bone wedges, the body's own bone material is removed preferably from the pelvis by knocking-in a suitable punch sleeve. Such punch sleeves are known for removing preferably circular bone cylinders of a certain length and diameter. A bone piece or bone cylinder removed in this manner is subsequently sawn into a wedge shape. According to the invention, for cutting up the bone or bone pieces there is provided a device or saw jig which permits the manufacture of bone wedges of a defined size and defined angle.

The device according to the invention comprises at least one mounting part in which there is formed a receiving channel for accommodating a bone piece. In this region of the receiving channel in the mounting part there is formed at least one slot running obliquely to the longitudinal axis of the receiving channel. For cutting a bone wedge to size, a previously removed bone piece, preferably a bone cylinder, is applied into the receiving channel of the mounting part. A saw blade, preferably according to the principle of a reciprocating saw, may be introduced through the slot formed in the mounting part, wherein the bone is cut to size into a predetermined, defined wedge shape. One may set the desired wedge shape by way of the selection of the angle between the slot and the longitudinal axis. In this manner the device according to the invention in a very simple and defined manner permits the cutting to size of bone wedges required with adjustment osteotomy for aligning the bones. At the same time one may produce two identical bone wedges, in particular in one working procedure. The receiving channel preferably has a width which corresponds to the size of a bone piece to be accommodated. In this manner the bone piece may be securely fixed in the receiving channel in the direction transverse to the longitudinal axis of the receiving channel and of the bone piece, so that by way of the slots in the mounting part one may cut the bone piece into a defined wedge shape without there existing the danger of the bone piece slipping relative to the slots. The width of the receiving channel is preferably directed to the outer diameter of a bone cylinder to be accommodated so that this may be accommodated without play in the direction transverse to the longitudinal axis.

Preferably two intersecting slots are provided which in each case run at an acute angle to the longitudinal axis of the receiving channel. By way of this arrangement of the slots which intersect or are connected to one another at a point, it is possible to be able to produce different cutting angles with one saw jig. For this the saw blade may alternatively be guided through one of the two intersecting slots. The point of intersection of both slots preferably lies in the longitudinal axis of the receiving channel and further preferred in the middle of the longitudinal axis. Thus the bone piece or the bone cylinder may be optimally exploited in order to produce two identically formed bone wedges.

In particular with the arrangement of intersecting slots the angle between the slots and the longitudinal axis of the receiving channel is preferably in each case between 4° and 13°. Angles of 5.5° and 7.5° as well as 9.5° and 11.5° are particularly preferred, wherein also other larger or smaller angles are possible depending on the extent of the correction to be carried out. Bone wedges with a differing gradient or differing angle are required according to the intensity of the malposition of the bones to be corrected. At the same time one may make available different devices or saw jigs with differing angles between the slots, in order to be able to cut to size a fitting bone wedge for each malposition to be corrected. One then merely needs to select the correct saw jig according to the size of the malposition to be corrected.

In a further preferred embodiment form the two slots run at different angles to the longitudinal axis of the receiving channel. In this manner one may produce bone wedges with different wedge angles. For example the one slot may have an angle of 5.5° to the longitudinal axis and the other intersecting slot an angle of 7.5° to the longitudinal axis. Alternatively 9.5° and 11.5° are conceivable for correcting larger malpositions.

In a particularly preferred embodiment form there is provided a second mounting part in which there is formed at least one slot congruent to the slot of the first mounting part. This second mounting part serves for fixing the bone piece in the first mounting part. After the bone piece or the bone cylinder is inserted into the receiving channel of the first mounting part and is aligned in this, the second mounting part is placed onto the first mounting part by which means the bone piece is clamped between the two mounting parts. In this arrangement the slots in the first and the second mounting parts are congruent to one another so that a saw blade may be simultaneously guided through the slots of the first and the second mounting part in order to cut the inserted bone piece into the desired wedge shape. By way of the fact that the saw blade is guided through the slots of both mounting parts, one achieves a more precise guiding of the saw blade and thus a more exact cut.

In the case that two slots are provided in the first mounting part, preferably the second mounting part too comprises two corresponding slots which are congruent to the slots in the first mounting part. One may therefore achieve an optimal guiding of the saw blade also for various wedge angles, i.e. with the use of each slot.

It is further preferred in each case to form one receiving channel in both mounting parts on surfaces facing one another. This design permits an even more precise fixation of the bone piece between the two mounting parts and thus relative to the slots formed in the mounting parts.

The receiving channels are preferably formed as a groove with a V-shaped cross section. An applied bone cylinder thus comes into line contact with the surfaces of the V-shaped groove. The V-shaped grooves preferably have an opening angle of 90°, i.e. their surfaces inclined to one another enclose essentially a right angle. One may also securely accommodate bone cylinders with a diameter which is not constant or with large tolerances in the V-shaped grooves, since the bone cylinder only comes into point or line contact with the groove and does not bear on its surface over its complete circumference. The surfaces of the receiving channel or of the receiving channels are also preferably roughened in order to prevent slippage of the bone cylinder in the direction of its longitudinal axis or the longitudinal axis of the receiving channel. One thus achieves a secure fixation of the bone cylinder relative to the slots.

It is useful to provide guide elements on both mounting parts, which position the mounting parts to one another such that the slots and the receiving channels of both mounting parts are arranged facing one another and congruently. The guide elements thus serve the automatic alignment of the two mounting parts to one another, by which means the use of the device according to the invention is simplified.

Preferably the guide elements extend normally to the longitudinal axis of the receiving channel and normal to the surface of the mounting part with the receiving channel, and the two mounting parts are movable linearly to one another, guided in their longitudinal direction. After insertion of the bone part into the receiving channel on the first mounting part the second mounting part is placed onto the first mounting part, wherein it is guided by the guide elements. The movability of the mounting parts in the direction of the guide elements ensures a secure fixation of the bone piece between the two mounting parts. The outer contour or the outer diameter of the bone pieces or the bone cylinder perhaps have greater tolerances, wherein however the movability of the mounting parts in the direction of the guide elements permits the accommodation of bone pieces with a differing diameter, i.e. with a slight conical shape.

The guide elements are preferably designed in a manner such that grooves are formed on two side surfaces of the first mounting part which are opposed to one another, said grooves being able to be brought into engagement with corresponding tabs of the second mounting part, wherein the tabs and the grooves extend in one direction normally to the longitudinal axis of the receiving channel and normally to that surface of the mounting part with the receiving channel. If the second mounting part is placed onto the first mounting part, the tabs of the second mounting part engage laterally around the first mounting part, by which means one achieves a guiding transverse to the longitudinal axis of the receiving channel. The tabs simultaneously engage into the grooves so that one likewise achieves a guiding in the longitudinal direction of the receiving channel and the slots in both mounting parts are aligned congruently. The tabs due to an arcuate or convex design of their side edges allow the first mounting part and the second mounting part to be able to be tilted to one another so that at one longitudinal end they have a smaller distance to one another that at the opposed end. This also allows conical bone pieces to be able to be held securely between the two mounting parts.

### BRIEF DESCRIPTION OF THE DRAWINGS

Hereinafter the invention is described by way of example and by way of the accompanying figures. There are shown in:

FIG. 1 a plan view of the first mounting part,

FIG. 2 a lateral view of the mounting part in the direction of the arrow 11 in FIG. 1,

FIG. 3 a plan view of the second mounting part,

FIG. 4 a lateral view of the mounting part according to FIG. 3 in the direction of the arrow IV and

FIG. 5 a perspective view of the assembled mounting parts.

### DETAILED DESCRIPTION OF THE INVENTION

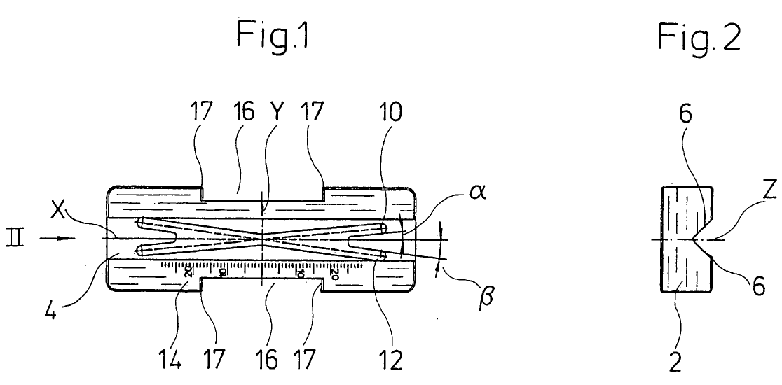

FIG. 1 shows a plan view of the first, lower mounting part 2. A V-shaped receiving groove extends in the direction of the longitudinal axis X on the upper side for accommodating a bone piece. The side walls 6 (see FIG. 2) of the receiving groove 4 preferably extend at an angle of essentially 90° to one another. In the region of the receiving groove, two continuous slots 10 and 12 proceeding from the intersection point of the longitudinal axis X and the transverse axis Y extend in the direction normal to the axes X and Y through the lower mounting part 2. The two slots 10 and 12 intersect at the intersection point of the axes X and Y, i.e. they run crossed to one another. The slots 10 and 12 run at an angle a, β to the longitudinal axis X. In the shown example the angle a between the slot 10 and the longitudinal axis X is more acute or smaller than the angle β between the slot 12 and the longitudinal axis X. This permits the manufacture of bone wedges with a different gradient or with a different wedge angle according to which of the two slots 10 and 12 is used for guiding the saw blade. Proceeding from the transverse axis Y, a scale 14 is attached on the surface of the lower parts 2 in both directions along the X-axis, said scale simplifying the centric insertion of the bone piece with respect to the axis Y so that one may manufacture two identical bone wedges. On both longitudinal sides of the lower mounting part 2 there are formed recesses or grooves 16 which extend in a direction normal to the axes X and Y. The side surfaces in the region of the grooves 16 at the same time run parallel to the axis X. The recesses or grooves 16 serve for guiding the second mounting part, as will be explained later.

FIG. 2 shows a lateral view of the lower mounting part 2 in the direction of the arrow 11 in FIG. 1 in which the V-shaped shape of the receiving channel may be recognised. The surfaces 6 run symmetrically, essentially at an angle of 45° to the co-ordinate axis Z which extends normally to the longitudinal axis X and to the transverse axis Y.

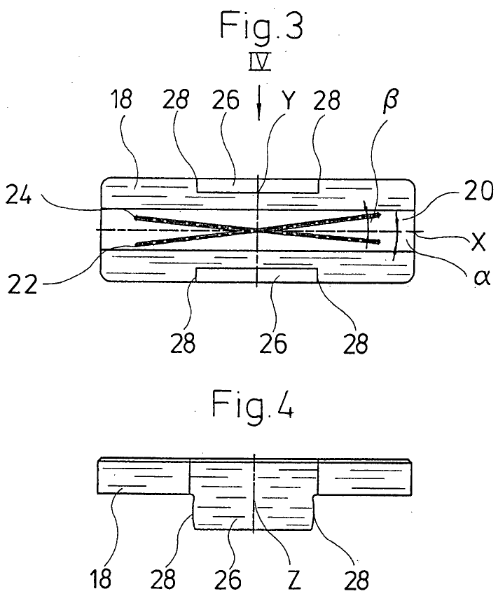

FIG. 3 shows a plan view of the second, upper mounting part 18. Also in the mounting part 18 on one surface there is formed a V-shaped groove as a receiving channel 20 extending in the direction of the longitudinal axis X. In the region of the receiving channel 20 there are arranged slots 22 and 24 corresponding to the lower mounting part 2 according to FIG. 1, which run crossed to one another and intersect at the intersection point of the axes X and Y. The slots 22 and 24 run as the slots 10 and 12 at an angle a and β to the longitudinal axis. This allows the slots 22 and 24 to be applied congruently onto the slots 10 and 12 if the mounting parts 2 and 18 are placed on one another. Proceeding from the surface of the mounting part 18 in which the receiving channel 20 is formed, lateral tabs 26 at the same time extend parallel to the longitudinal axis X. The side surfaces of the tabs 26 are formed such that they may enter into the grooves 16 on the lower mounting part 2, wherein the inner surfaces of the two tabs 26 which face one another come to bear on the opposed outer side surfaces of the lower mounting part 2 in the region of the grooves 16 and thus align the upper mounting part 18 with respect to the lower mounting part 2 in the direction of the axis Y. In the direction of the axis X the width of the tabs 26 is directed to the width of the grooves 16 in this direction so that an alignment in the direction of the axis X is likewise effected if the upper mounting part 18 is placed onto the lower mounting part 2. The end-face edges 28 of the tabs 26 extending parallel to the axis X are formed rounded or curved, as may be recognised in FIG. 4. The end-face edges 28 come into contact with the end-faces 17 in the grooves 16 if the tabs 26 are applied into the grooves 16. By way of the fact that the end-faces 28 are formed curved, the mounting parts 2 and 18 if they are applied onto one another may be tilted to one another in a plane tentered by the axes X and Z so that slightly conical bone pieces may also be securely fixed between the receiving grooves 24.

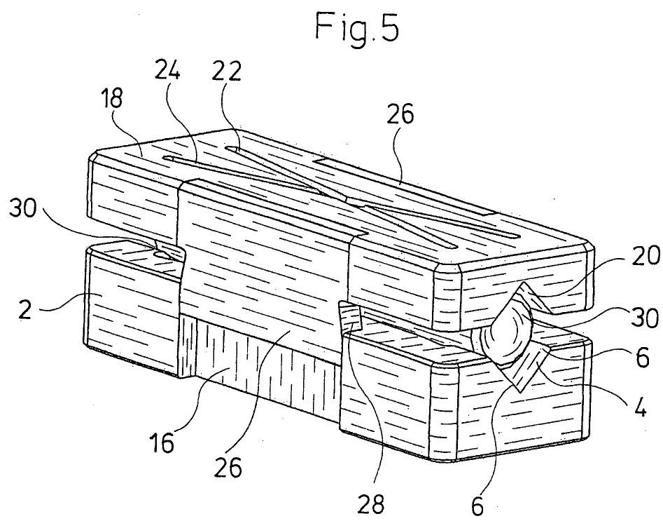

The assembled condition of the mounting parts 2 and 18 with the inserted bone piece 30 is explained by way of FIG. 5. Firstly an essentially circularly cylindrical bone piece 30 which has been previously punched out of preferably the pelvic bone is applied into the receiving channel 4 on the lower mounting part 2, wherein the bone piece 30 comes into line contact with the surfaces 6 of the receiving channel 4. At the same time the bone piece 30 in the receiving channel 4 may be aligned with the help of the scale 14 as explained by way of FIG. 1. Subsequently the upper mounting part 18 is placed onto the lower mounting part 2 in a manner such that the tabs 26 enter into the grooves 16 at the sides of the lower mounting part 2. The receiving channels 4 and 20 each have a depth which is less than half the diameter of the bone piece 30 to be accommodated. It is thus achieved that the mounting parts 2 and 18 are distanced from one another also with an inserted bone piece 30 and in this manner bone pieces 30 with a slightly varying or non-exact cylinder shape may be securely held. At the same time the bone piece 30 likewise comes into linear contact with the oblique side surfaces of the receiving channel 20 in the upper mounting part 18. On account of the curved end-face edges 28 of the tabs 26 the upper mounting part 18 and the lower mounting part 2 may be tilted to one another such that at a longitudinal end the mounting parts 2 and 18 lie closer together than at the opposed longitudinal end (in the direction of axis X). This allows slightly conical bone pieces or bone cylinders 30 to be clamped between the mounting parts 2 and 18.

If the bone piece 30 is fixed between the mounting parts 2 and 18 in this manner, one may introduce a saw blade through the slots 22 and 24 and thus likewise through the slots 10 and 12 congruently below this, and at the same time the bone piece 30 may be simultaneously cut to size into two wedge-shaped pieces with identical wedge angles. Two identical bone wedges with predefined wedge angles may be formed very simply in this manner. For forming bone wedges with different wedge angles one may keep various saw jigs in each case consisting of a lower mounting part 2 and an upper mounting part 18, wherein the saw jigs differ in the angles a and β of the slots 10,12 and 22, 24 with respect to the longitudinal axis X.

The bone wedges produced in this manner are particularly applied in a new method for correcting malposition of the bones, in particular knock-knees and/or bowlegs. With this method one firstly saws incisions at defined locations in the region of the arcuate bone, and the premanufactured bone wedges are applied into these incisions for aligning the bone, by which means the bone may be straightened. This method has the advantage that no fixation means needs to be employed for fixing the aligned bone. Later operations to remove these fixing means are thus no longer necessary. Since the wedges may be manufactured from the body's own bone material, a good and rapid growing together of the aligned bone is further possible.

### List of Reference Numerals

2 lower mounting part

4 receiving channel

6 side surfaces

10,12 slots

14 scale

16 grooves

17 end-face walls

18 upper mounting part

20 receiving channel

22,24 slots

26 tabs

28 end-face edges

30 bone piece

1. A device for cutting bones to size, in particular with displacement osteotomy, with at least one mounting part (2) in which there is formed a receiving channel (4) for accommodating a bone piece (30) and in which in the region of the receiving channel (4) there is formed at least one slot (10, 12) arranged at an angle obliquely to the longitudinal axis of the receiving channel.

2. A device according to claim 1, with which there are provided two intersecting slots (10, 12) which each run at an acute angle to the longitudinal axis (X) of the receiving channel (4).

3. A device according to claim 1 or 2, with which the two slots (10, 12) run at different angles (a, β) to the longitudinal axis of the receiving channel.

4. A device according to one of the preceding claims, with which there is provided a second mounting part (18) in which there is formed at least one slot (22,24) congruently to the slot (10,12) of the first mounting part (2).

5. A device according to claim 4, with which in the second mounting part (18) there are formed two slots (22,24) congruent to the slots (10, 12) of the first mounting part (2).

6. A device according to claim 4 or 5, with which in both mounting parts (2, 18) on surfaces facing one another there is in each case formed a receiving channel (4, 20).

7. A device according to one of the preceding claims, with which the receiving channels (4, 20) are each formed as a groove with a V-shaped cross section.

8. A device according to one of the preceding claims, with which the surfaces of the receiving channel (4, 20) are roughened.

9. A device according to one of the preceding claims, with which on the two mounting parts (2, 18) there are formed guide elements (16, 26) which position the two mounting parts (2, 18) to one another such that the slots (10, 12, 22, 24) and the receiving channels (4, 20) of both mounting parts (2, 18) face one another and are arranged congruently.

10. A device according to claim 9, with which the guide elements (16, 26) extend normally to the longitudinal axis (X) of the receiving channel (4, 20) and normally to the surface of the mounting part (2, 18) with the receiving channel (4, 20), and the two mounting parts (2, 18) are movable linearly to one another guided by the guide elements (16, 26) in their longitudinal direction (Z).

11. A device according to claim 9 or 10, with which on two opposed side surfaces of the first mounting part (2) there are formed grooves (16) which may be brought into engagement with corresponding tabs (26) of the second mounting part (18), wherein the tabs (26) and grooves (16) extend in a direction (Y) normal to the longitudinal axis (X) of the receiving channel (4) and normal to that surface of the mounting part (2) with the receiving channel (4).
